(19) United States
(12) Patent Application Publication
(10) Pub. No.: US 2015/0114911 A1
(43) Pub. Date: Apr. 30, 2015

(54) DEVICE FOR STERILISING WATER

(71) Applicant: Ian Stewart HELMORE, Hertfordshire (GB)

(72) Inventor: Ian Stewart Helmore, Hertfordshire (GB)

(21) Appl. No.: 14/399,576

(22) PCT Filed: May 9, 2013
(86) PCT No.: PCT/GB2013/051202

§ 371 Date: Nov. 7, 2014

(30) Foreign Application Priority Data
May 10, 2012 (GB) 1208224.4

(51) Int. Cl. C02F 1/32; A61L 2/10

(52) U.S. Cl. CPC C02F 1/32; A61L 2/10; C02F 2103/003
USPC 210/748.11; 250/435

(57) ABSTRACT

A tap for sterilising water, the tap comprising an inlet, a source of ultraviolet radiation, a sterilisation zone and an outlet, the outlet consisting of one aperture through which the water exits the device, wherein the source of UV radiation is positioned in the sterilisation zone such that substantially all of the internal surfaces of the outlet portion can be directly irradiated by the source of ultraviolet radiation, and wherein the source of ultraviolet radiation and the aperture are arranged such that no ultraviolet radiation can be transmitted directly from the source of ultraviolet radiation through the aperture to leave the device.

The present invention relates to a device for sterilising water, and in particular to a tap for sterilising water using ultraviolet radiation.

It is known that bacteria and other microorganisms can be killed by the use of electro-magnetic radiation such as ultraviolet radiation. In particular, ultraviolet radiation may be used to sterilise a flow of water by passing the water near to a source of ultraviolet radiation for a sufficient time for any microorganisms to be killed. This can be performed in a batch or a continuous manner. Taps, otherwise known as faucets or spigots, are used in many different environments to provide a flow of fluid, and in particular a flow of water. There are many different designs of tap depending on the particular use and location in each case.

Hygiene can be a concern in some areas, particularly where a large number of different people use the same tap. This can lead to the presence of microorganisms such as bacteria on the tap. This is routinely addressed by regular cleaning of the facilities, by using chemical disinfectants. However this behaviour has shortcomings which the present invention seeks to address.

According to the present invention there is provided a tap for sterilising water, the tap comprising an inlet, a source of ultraviolet radiation, a sterilisation zone and an outlet, the outlet consisting of one aperture through which the water exits the device, wherein the source of UV radiation is positioned in the sterilisation zone such that substantially all of the internal surfaces of the outlet portion can be directly irradiated by the source of ultraviolet radiation, and wherein the source of ultraviolet radiation and the aperture are arranged such that no ultraviolet radiation can be transmitted directly from the source of ultraviolet radiation through the aperture to leave the device.

Preferably, the outlet portion comprises an elongate tap head.

Conveniently, the outlet portion comprises a cylindrical channel for the water.

Advantageously, the outlet portion comprises a cylindrical channel for the flow of water which has an internal diameter which is greater than the internal diameter of the aperture.

Preferably, the tap provides laminar flow of the water through the aperture.

Conveniently, the aperture is located in a concave portion of the exterior surface of the tap.

Advantageously, the aperture has an internal diameter of from 7 to 12 millimetres, preferably about 9 millimetres.

Preferably, water is retained within the tap when the water is not flowing.

According to an aspect of the invention, there is provided a method of sterilising a flow of water comprising providing a tap as defined in any of the preceding paragraphs, flowing water into the tap, through the sterilisation zone and out of the aperture.

According to another aspect of the invention, there is provided a method of sterilising a tap comprising providing a tap as defined in any of the preceding paragraphs, and exposing the external surface of the tap to ultraviolet radiation.

Preferably, the internal surface of the tap is irradiated with ultraviolet radiation simultaneously with the irradiation of the external surface of the tap with ultraviolet radiation.

The present invention will now be described, by way of example, with reference to the following drawings, in which;

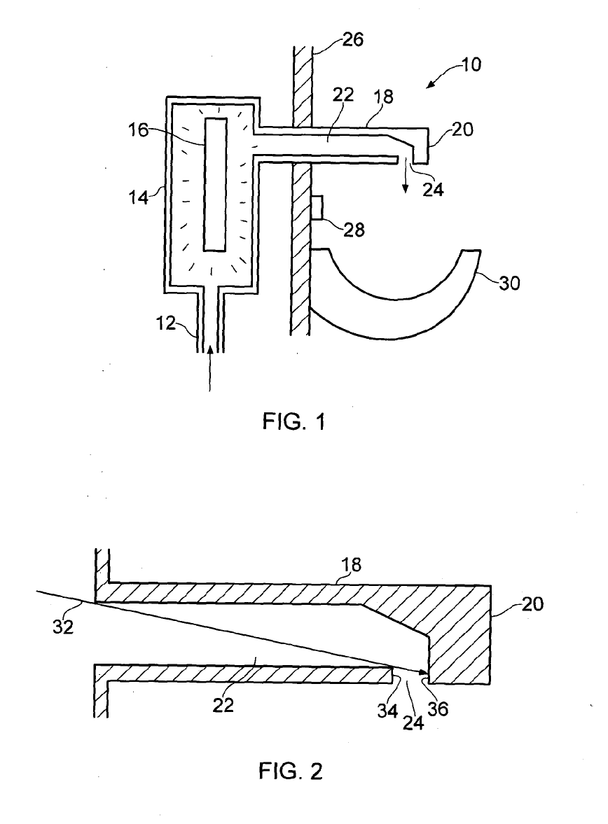

FIG. 1 is a schematic view of a tap,

FIG. 2 is a cross-section of a tap head,

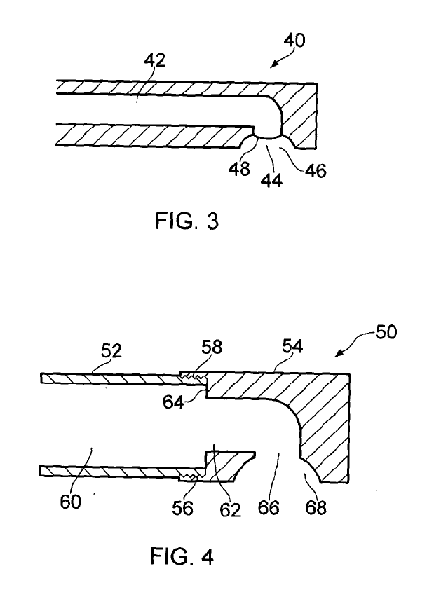

FIG. 3 is a cross-section of a tap head,

FIG. 4 is a cross-section of a tap head,

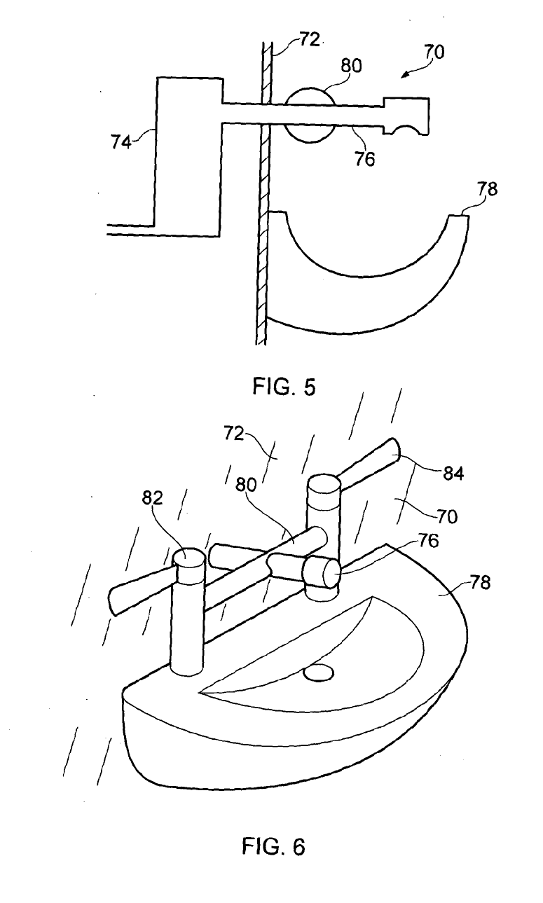

FIG. 5 is a cross-section of a tap and sink,

FIG. 6 is a perspective view of a tap and sink,

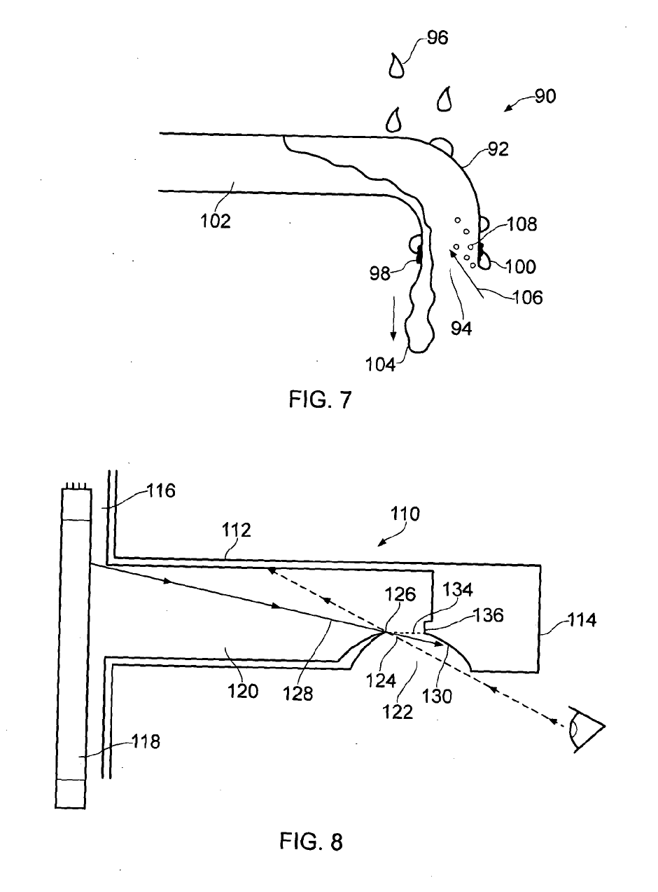

FIG. 7 is a schematic view of a prior art tap,

FIG. 8 is a cross-section of a tap head,

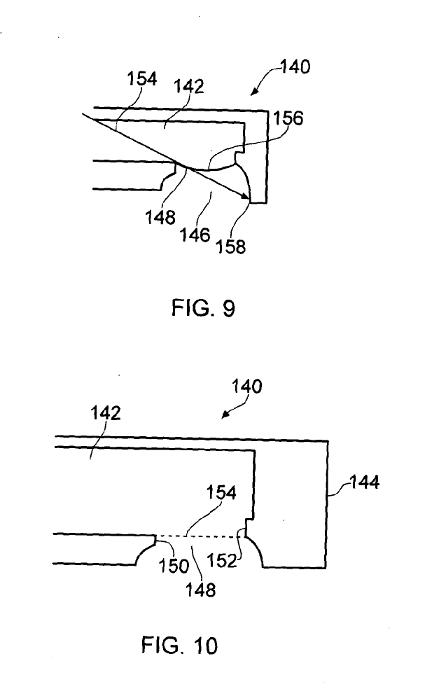

FIG. 9 is a cross-section of a tap head,

FIG. 10 is a cross-section of a tap head,

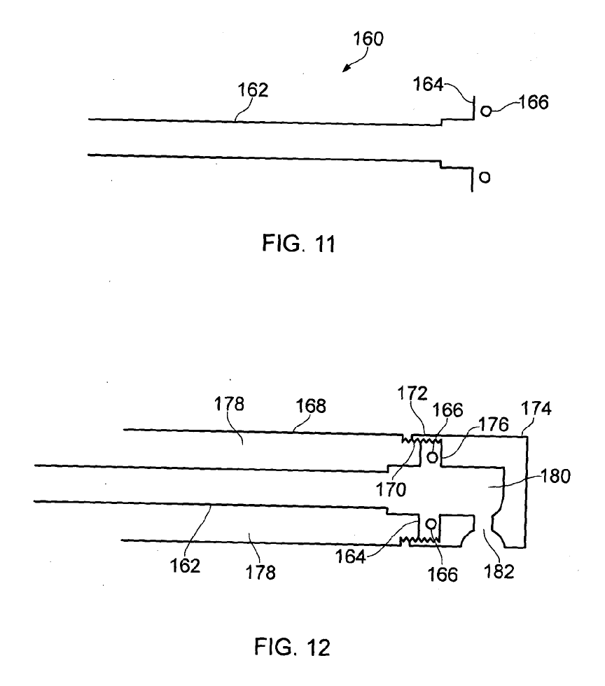

FIG. 11 is a cross-section of a pipe fitting,

FIG. 12 is a cross-section of a tap head,

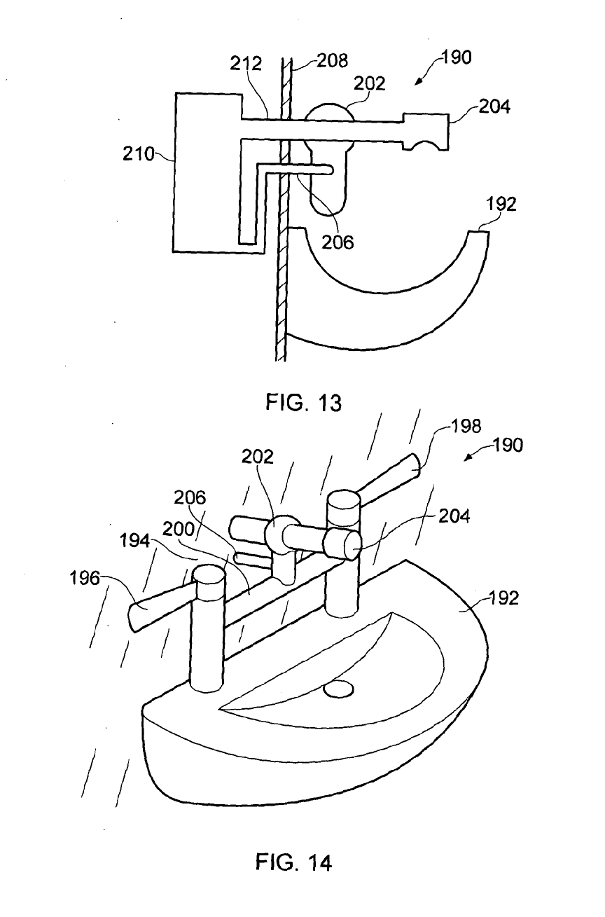

FIG. 13 is a cross-section of a tap and sink, and

FIG. 14 is a perspective view of a tap and sink,

The invention relates to outlets for liquids and address the problem of contamination by microorganisms. In many situations, liquid outlets, such as water taps, can be contaminated with microorganisms. The microorganisms can be present on an external surface of the tap, and/or in the interior of the tap. Some microorganisms can be harmful to humans, and can cause a user of the tap to become infected, for example when they wash their hands. The present invention addresses this problem by trying to eliminate microorganisms on an external surface of the tap, and/or inside the tap.

One particular area of concern is nosocomial infections, otherwise known as hospital-acquired infections. Patients can become infected by microorganisms present in a healthcare facility, leading to health problems that were unrelated to the original illness of that patient. Unfortunately, nosocomial infections can be fatal in some cases. There are many microorganisms that cause these infections, including legionella bacteria, Pseudomonas, and coliforms such as Escherichia coli.

It is known that some microorganisms can be present within the infrastructure of buildings, especially in water supply systems. There are various methods used to control this problem, such as heating water to high temperature and circulating the hot water around the system. Periodic chemical treatments are also used to eliminate microorganisms present, and ultraviolet (UV) radiation is also used within water supply systems. While these systems can eliminate microorganisms for a time, or from a certain part of the supply system, the systems are routinely re-colonised by microorganism over time.

Microorganisms are present in the environment and often enter a water supply system via an outlet, such as a tap. The presence of microorganisms in and/or on a tap can lead to users becoming infected.

In FIG. 1 a tap (10) is shown which comprises a system in which water enters the tap, is sterilised by ultraviolet radiation and leaves the tap. Water enters the system via an inlet (12) which leads to a sterilisation chamber (14). The sterilisation chamber (14) contains a source of ultraviolet radiation (16). The source of ultraviolet radiation produces high energy electromagnetic radiation in the ultraviolet part of the spectrum which kills or deactivates microorganisms. Preferably, the source of ultraviolet radiation is a gas discharge tube that can produce germicidal ultraviolet radiation. A tap head (18) leads from the sterilisation chamber (14), terminating in a distal end (20). Near the distal end (20) of the tap head (18) there is located an aperture (24). A channel (22) is located within the tap head (18) and provides a path for water to flow from the sterilisation chamber (14) to the aperture (24).

In this embodiment, the tap is located through a wall, so that the sterilisation chamber (14) is located behind the wall (26), with the tap head (18) projecting through the wall (26), terminating over a sink (30). A sensor (28) is provided near the tap, so that a user can turn the flow of water on without physically touching any components.

In use, water can flow through the inlet (12) into the sterilisation chamber and is then exposed to ultraviolet radiation by the source of ultraviolet radiation (16). The water then flows through the channel (22) inside the tap head (18) towards the aperture (24), and then leaves the device through the aperture (24). The flow of water and the power of the UV source (16) are arranged to ensure that a sufficiently high level of sterilisation is achieved. The source of ultraviolet radiation (16), the internal channel (22) and the aperture (24) are arranged such that practically all of the internal surfaces of the channel (22) are directly exposed to ultraviolet radiation. This ensures that any microorganisms present within the channel are killed or inactivated. The source of ultraviolet radiation (16), the channel (22) and the aperture (24) are also arranged so that no ultraviolet radiation can be transmitted directly from the source (16) through the aperture (24). This is important because ultraviolet radiation can be harmful to users, and is not visible to the human eye. In practice, a small part of the device near the aperture (24) may be in UV shadow. However, the design of the tap ensures that this is kept to a practical minimum.

Turning to FIG. 2, the tap head (18) is shown in cross-section. This shows that the tap head (18) contains a substantially cylindrical channel (22) along the axis of the elongate tap head. Towards the distal end (20) of the tap head (18) the channel (22) tapers towards the downward facing aperture (24). Preferably, the aperture in the tap; is circular. As shown, ultraviolet radiation (32) is transmitted along the channel (22) from the source of radiation (16), exposing practically all of the internal surface of the channel (22) with ultraviolet radiation. The aperture (24) is arranged such that no UV radiation can be transmitted out of the aperture to expose a user to potentially harmful radiation. This is achieved by placing the aperture (24) at a glancing angle relative to the source of the ultraviolet radiation, so that any UV radiation entering within the aperture terminates on the internal wall (36) which defines the aperture (24). In practice this means that there is a small amount of the wall (36) which defines the aperture (24) which is not directly exposed to UV radiation. The area of UV shadow (34) is kept to a practical minimum.

It is preferred that the internal surface area of the channel that is in UV shadow (34) is less than 25% of the area defined by the aperture. More preferably, the area in UV shadow is less than 20%, and more preferably less than 10% of the area that is defined by the aperture.

For example, in one embodiment the internal diameter of the channel near the aperture is around 16 mm, and the diameter of the aperture is around 9 mm. The area of the internal surface area surround the aperture (24) that is in UV shadow (34) extends no more than 2 mm from the external surface of the tap head (18) into the aperture (24). In general, the UV shadow extends no more than about 5 mm from the external surface of the tap, preferably no more than about 4 mm, and most preferably, no more than about 2 mm.

This ensures that the maximum surface area within the tap is directly illuminated with germicidal radiation, but ensures that no harmful radiation is directly transmitted through the aperture (24).

FIG. 3 is a similar view to FIG. 2 showing a tap head (40) of similar unitary construction to the tap head (18) in FIG. 2. The tap head (40) comprises a cylindrical unitary design comprising an internal channel (42) with a flow of water, leading to an aperture (44) at the distal end of the tap head (40). In this case there is a concave region (46) (or recess) on the exterior of the tap head (40). Thus, the aperture (44) leads to the concave area (46) on the surface of the tap (40). In the embodiment shown, the tap head (40) contains water which is not flowing. In this situation, the water has formed a meniscus (48) across the aperture (44).

The design of the tap head (40) encourages the water to remain within the channel (42) when the flow of water is turned off. This is useful in order to minimise re-colonisation of the interior of the tap by microorganisms that are present in the ambient environment. Microorganisms could enter the channel (42) if the water drained out of the tap when not in use. This would leave a moist environment in which microorganisms could thrive. This is a problem which is endemic in current water systems, including the frequent re-colonisation of water systems by microorganisms, including legionella and pseudomonas. By helping to maintain water within the interior of the tap when the water is not flowing, the invention minimises the risk of re-infection of the water system.

FIG. 4 shows a different design to that shown in FIG. 3. FIG. 4 shows a tap head (50) which has a two-piece design, rather than unity design. The tap head (50) comprises a proximal portion (52) and a distal portion (54). The proximal portion (52) has a substantially cylindrical shape, terminating in a portion having an external screw thread (56). Distal portion (54) has a portion sized to receive the proximal portion (52), which is provided within an internal thread (58). In use the thread (56) of the proximal portion (52) engages with the thread (58) of the distal proportion (54). In doing so, the proximal portion (52) abuts against an internal surface (64) of the distal portion (54). The distal portion (54) is provided with an interior channel (62), which in the embodiment shown is of a smaller internal diameter than the channel (60) in the proximal portion (52). The channel (62) of the distal portion leads to an aperture (66), which is located within a concave portion (64) of the external surface of the distal portion (54). Although the distal portion (54) and the proximal portion (52) can be simply screwed together, it is preferred that a locking composition (preferably food grade) is used to ensure a secure fit.

FIG. 5 shows a tap (70) installed behind a wall (72). The arrangement is similar to that shown in FIG. 1 except that the flow of water is manually controlled, rather than using a sensor. FIG. 5 shows the tap (70) comprising a sterilisation chamber (74) located behind a wall (72). A tap head (76) leads from the sterilisation chamber (74), through the wall (72), to end above a sink (78). As suppressing through the wall (72), the tap head (76) also passes through a member (80), which will be described below with reference to FIG. 6.

FIG. 6 is a perspective view of the tap (70) which is shown in FIG. 5. FIG. 6 shows the tap head (76) projecting from a wall, through a member (80) to terminate above a sink (78). The member (80) joins to controls (82) and (84). The user can swivel the controls (82) and (84) with their hands or elbows to control the flow of hot and cold water through the tap (76). The arrangement is meant to look similar to traditional taps used in many settings, for example in hospitals. The controls (82) and (84) can directly control valves which adjust the flow of hot and cold water which enter the sterilisation chamber (74). Alternatively, they could control the flow of water using electronic sensors. In a preferred embodiment, the water valves are controlled by flexible cables. This allows the control cables to be bent in angles (e.g. 90°) and then pass through the wall to control valves that are locate behind the wall.

An important feature of the invention is that the taps are provided with a single aperture. This prevents the formation of areas which are not directly irradiated with ultraviolet radiation, and which could form areas for microorganisms to thrive. So the taps of the invention do not contain any inserts, roses, aerators or diffusers or any other form of flow control device in the aperture. This not only helps to avoid any areas in which microorganisms could live, but also helps to produce laminar flow. Laminar flow is useful in that it allows water to flow through and out of the tap without causing splashes or droplets. Splashing is unwanted in many circumstances, particularly in hospital situations.

The tap can be formed of any suitable material which is resistant to ultraviolet radiation. Preferably stainless steel is used.

The tap head can also be removed from the ultraviolet sterilisation chamber for sterilisation in an autoclave. This involves extended heating of the tap head in order to ensure sterilisation.

As shown in the Figures, the exterior of the tap has a design which minimises any sites in which a microorganism could live. The exterior surface of the tap is preferably a smooth surface with no cavities which could harbour microorganisms. Preferably, the external surface of the tap is sterilised using UV radiation. For example, the entire external surface area of the tap head could be exposed to ultraviolet radiation for a sufficient time as to kill or inactivate any microorganisms present to a desired level of sterility. Such a method would involve exposing the total exterior surface area of the tap head to ultraviolet radiation, including irradiating ultraviolet radiation through the aperture and in to the interior of the tap. This would irradiate any area of the tap that was not directly irradiated by the internal source of UV radiation. Preferably, the sterilisation of the tap involves irradiating both the internal surface area of the tap head and the external surface area of the tap head.

FIG. 7 shows a cross-sectional view of a prior art tap 90, having a curved portion 92 leading to an outlet 94. A user will typically wet their hands with water, and then obtain soap from a dispenser. The user will then rub their hands with the soap to clean them, which can leads to drips of unclean soapy water 96 falling from their hands onto the tap 90. The unclean water 96 can contain bacteria and other germs from the person washing their hands, and can lead to the presence of such microorganisms 100 on the external surface of the tap. The presence of microorganisms on the surface of the tap is not desirable, and could lead to infection of other users either by touching the tap, or contacting water leaving the tap that picks up bacteria etc from previous users.

One potential problem is the introduction of such unwanted microorganisms from outside the tap to the interior of the tap. This could happen if water containing microorganisms is brought from the exterior of the tap into the interior. In FIG. 7, a drop of water 100 is shown on the outside of the tap near the outlet aperture 94. After the tap is turned off by the use, water 102 that is present inside the tap will drain out of the hole 94, shown as a stream of water 104. Simultaneously, a stream of air 106 will be drawn into the tap to replace the volume of draining water. This intake of air may entrain unclean water, for example the drop 100 of unclean water may be drawn into the interior of the tap in the form of small droplets 108. If bacteria or other microorganisms are present in the entrained water, then they could live in the interior of the tap, perhaps colonising the interior tap surface. This could lead to the formation of persistent biofilms, which can be difficult to eliminate.

So, it can be seen that traditional liquid outlets, such as a water tap, can be a source of infection because of the presence of microorganisms on the exterior and/or interior of the tap. People can become infected by touching the tap, or just by washing.

FIG. 8 shows an embodiment of the tap 100 of the invention which addresses these problems. The tap 110 comprises a tap head 112 of generally cylindrical shape extending from a sterilisation chamber 116 and terminating in a distal end 114. A UV lamp 118 is positioned within the sterilisation zone 118. The tap head 112 contains a generally cylindrical interior channel 120 extending away from the sterilisation zone. UV radiation can be transmitted directly from the UV lamp 118 to all of the interior surface of the channel 120 to eliminate microorganisms.

In a similar way to other embodiments, the tap head 112 has a concave portion or recess 122 near the distal end 114 which, in use, is positioned on the downward facing side of the tap 110. The channel 120 is connected to the recess 122 by a single aperture 124. Water can flow through the sterilisation chamber or zone 116, past the UV lamp 118, and along the channel 120, whilst being directly irradiated with germicidal UV radiation. The water will then flow out of the aperture 124.

In the embodiment shown, the proximal edge 126 of the aperture 125 is very thin. In other words, the edge 126 of the aperture 125 nearest the source of ultraviolet radiation is sharp. This has the effect that the edge of the tap head that defines the aperture 124 is not in UV shadow, i.e. it is directly illuminated with UV radiation from the UV lamp 118. The use of a sharp edge to define at least a portion of the aperture is a general preferred feature of the invention.

A dotted line 132 shows the level of water when the tap is not is use, i.e. when the interior is full of water which is not flowing out of the tap. The dotted line 132 extends from the sharp edge 126 of the aperture to the distal side 134 of the aperture. As shown, the water level 132 extends across the aperture 124 at its intersection with the recess 122.

The embodiment shown in FIG. 8 has another preferred feature, in that some UV radiation is transmitted directly from the UV lamp 118 through the aperture 122 to maximise germicidal effects. The ray 128 in FIG. 8 shows the further possible extent of directly transmitted UV radiation from the lamp 118 through the aperture 123. The ray 126 terminates on the surface of the tap head 112 within the recess 130. This means that the UV radiation that is transmitted through the aperture 122 is prevented from leaving the recess 122 itself. This helps to ensure the any bacteria etc in the vicinity of the aperture 124 are inactivated whilst preventing the user of the tap from being exposed to UV radiation. In the arrangement shown in FIG. 8, a user cannot see the UV lamp through the aperture 124.

FIG. 9 is a cross-sectional view of a tap head 140 of generally cylindrical shape containing an interior channel 142. As in other embodiments, water can flow through a UV radiation chamber and into the channel 142 whilst being irradiated. The water then flows along the channel 142 towards a distal end 144 of the tap head 140, and then leaves the tap via a single aperture 148, formed in a concave recess 146. The aperture 148 is substantially circular in this embodiment (although apertures of other shapes could be used), and is circumscribed by a short cylindrical-shaped portion of the tap, having a proximal surface 150 and a distal surface 152 (relative to the source of UV radiation). The level of the water when not flowing is shown as a dotted line 154, extending from the top of the proximal surface 150 and the bottom of the distal surface 152.

FIG. 10 shows the tap head 140 of FIG. 9 but with water inside the channel 142. The water is not flowing, and so remains within the interior channel 142, forming a meniscus 156 across the aperture 148. The water stays within the tap and so does not normally drain out of the tap when not in use. This prevents the introduction of air (potentially entraining microorganisms) into the interior of the tap.

As shown, the maximum extent of directly transmitted radiation is shown by a ray 154. The ray 154 passes through the aperture 148 and terminates on the distal surface 158 of the recess 146. This helps to ensure that any microorganisms present in the vicinity of the aperture 148 are inactivated by UV radiation whilst preventing users of the tap from being exposed to UV radiation.

FIG. 11 shows a pipe fitting 160 that can be used in conjunction with the tap heads of the invention. The pipe fitting 160 comprises a length of copper tubing 162 which terminates in a flared distal portion having an annular flange 164. An O-ring 166 is position on the distal surface of the flange 164. The pipe-fitting 160 can be used with a tap head as described herein, such as that shown in FIG. 4. This can be useful in situations where the use of UV radiation inside the tap is not feasible.

FIG. 12 shows the pipe-fitting 160 positioned inside a tap head of the invention, which comprises a tubular proximal portion 168 and a proximal portion 174. The pipe-fitting 162 is positioned inside the proximal portion 168 of the tap head. The proximal portion 174 has on its outer surface an external screw thread 170 near to its proximal end. The screw thread 170 can be connected to an internal screw thread 172 provided on the internal surface of the proximal end of the distal portion 174. Screwing the proximal portion 168 and distal portion 174 together brings the O-ring 166 into contact with an abutting surface 176 of the distal portion 174, forming a water-tight seal. Water can flow along the pipe-fitting 162 and into the interior 180 of the distal portion 174 of the tap head, and then out of an aperture 182. The annular space 178 between the pipe-fitting 160 and the proximal portion 168 of the tap head is dry, with water flowing along the pipe-fitting 160 and into the interior 180 of the distal portion 174 of the tap head.

If it is desired to use UV radiation to sterilise the interior of the tap head, it is possible to remove the pipe-fitting and install a sterilisation chamber and UV source as described above. This will add the benefit of inactivation of microorganisms within the tap, as well as the benefits that the structure of FIG. 10 offers in terms of ease of sterilisation of the exterior of the tap, and prevention of draining. Of course, other methods of inactivating microorganisms could be used to sterilise the water, such as periodic flushing and high temperatures).

As an alternate method of controlling water flow through to the UV chamber may be by means of feeding water through to the UV sterilisation chamber by the convenience of utilising commercially available standard tap fittings. FIGS. 13 and 14 show an example of this aspect.

FIGS. 13 and 14 show a tap 190 positioned above a sink 192. The tap uses commercially available tap fittings 194, which comprises hot and cold water supplies controlled by rotatable handles 196 and 198. The water is supplied by pipes that pass up each of the handle portions, and then pass along the connecting bar 200. In a conventional arrangement, the water would then pass up through a hole in the central bar 200 into a tap spout.

In the arrangement shown, the conventional tap spout has been removed and replaced by a tap 190 of the invention, using conventional connecting means. The connection can be made using means such as O-ring for making a water-tight seal, and a grub screw to hold the fittings in place. The tap 190 has a central column 202 which is inserted into the hole in the connecting bar 200, so that water is channeled into a pipe 206 which passes back through the wall 208. The pipe 206 leads the water into a UV sterilisation chamber 210 located behind the wall 208. The water is irradiated and sterilised as it passes through the chamber 210, and then enters the tap head 212 which leads back through the wall 208. The water then travels to the distal end 204 of the tap head and leaves via a single aperture, as discussed above.

This allows the benefits of the invention to be used with conventional tap fitting to reduce costs. It may also allow the invention to be retrofitted to existing water outlets that may already be in place.

A major advantage of the invention is that the tap heads are demountable. This means that the tap head can be removed and sterilised, preferably by being autoclaved. In practice, this would typically involve releasing the tap head from the UV sterilisation chamber (which would automatically turn off the source of UV). The tap head could then be taken away from that location to be sterilised. A replacement (sterilised) tap head could be installed at the same time.

It is to be appreciated that many of the features disclosed above can be beneficially used in devices other than taps, for example in showers and other types of water and fluid outlets.

1. A tap for sterilising water, the tap comprising an inlet, a source of ultraviolet radiation, a sterilisation zone and an outlet, the outlet consisting of one aperture through which the water exits the device, wherein the source of UV radiation is positioned in the sterilisation zone such that substantially all of the internal surfaces of the outlet portion can be directly irradiated by the source of ultraviolet radiation, and wherein the source of ultraviolet radiation and the aperture are arranged such that no ultraviolet radiation can be transmitted directly from the source of ultraviolet radiation through the aperture to leave the device.

2. A device as in claim 1, wherein the outlet portion comprises an elongate tap head.

3. A device as in claim 1, or 2, wherein the outlet portion is substantially cylindrical.

4. A tap according to any preceding claims wherein the outlet portion comprises a cylindrical channel for the water.

5. A tap according to any preceding claims wherein the outlet portion comprises a cylindrical channel for the flow of water which has an internal diameter which is greater than the internal diameter of the aperture.

6. A tap according to any preceding claim which provides laminar flow of the water through the aperture.

7. A tap according to any preceding claim wherein the aperture is located in a concave portion of the exterior surface of the tap.

8. A tap according to any preceding claim wherein the aperture has an internal diameter of from 7 to 12 millimetres, preferably about 9 millimetres.

9. A tap according to any preceding claim wherein water is retained within the tap when the water is not flowing.

10. A method of sterilising a flow of water comprising providing a tap as defined in any of the preceding claims, flowing water into the tap, through the sterilisation zone and out of the aperture.

11. A method of sterilising a tap comprising providing a tap as defined in any of claims 1 to 9, and exposing the external surface of the tap to ultraviolet radiation.

12. A method according to claim 11 wherein the internal surface of the tap is irradiated with ultraviolet radiation simultaneously with the irradiation of the external surface of the tap with ultraviolet radiation.

13. A tap substantially as hereinbefore described and/or as shown in the figures.
